(19) United States
(12) Patent Application Publication
(10) Pub. No.: US 2015/0257733 A1
(43) Pub. Date: Sep. 17, 2015

(54) WEARABLE IMAGING SYSTEM

(71) Applicant: Sonivate Medical, Inc., Beaverton, OR (US)

(72) Inventors: Scott Sutherland Corbett, III, Portland, OR (US); Kenneth Bates, Portland, OR (US); William McDonough, Portland, OR (US); Stephen B. Hooper, Bellingham, WA (US); Brian Epps, Edmond, WA (US)

(73) Assignee: Sonivate Medical, Inc., Beaverton, OR (US)

(21) Appl. No.: 14/204,978

(22) Filed: Mar. 11, 2014

(51) Int. Cl. A61B 8/00; A61B 8/08; A61B 8/14

(52) U.S. Cl. CPC A61B 8/4455; A61B 8/463; A61B 8/14; A61B 8/4427; A61B 8/462; A61B 8/4472; A61B 8/467; A61B 8/52

(57) ABSTRACT

A wearable ultrasound system comprising an ultrasound probe, a proximal wearable component electrically interconnected with said ultrasound probe adapted to be wearable on the hand, wrist, or arm of a user, and including at least one user interface mechanism, a processor, and one or more displays.

This invention was made with government support under contract number W81XWH-12-C-0194 awarded by the U.S. Army Medical Research and Material Command. The government has certain rights in the invention.

### BACKGROUND OF THE INVENTION

Ultrasound systems conventionally consist of two components: a probe and a workstation. The probe must contain the array of ultrasound transducer elements which convert electrical impulses to ultrasonic energy and vice-versa. Either the probe or the workstation includes front end functions such as beam forming, or creation of electrical impulses which are converted to and from ultrasonic energy by the transducer. The workstation contains a computational back-end, which processes the image data generated by the front end, a display, and a user interface including a keyboard or other means of input of user control.

These components are typically fairly large. If they are portable at all, they typically achieve that portability by being housed on a cart that can be wheeled from room to room in a clinic or hospital. Extremely portable systems tend to be laptop computer sized, and must be carried in a user's hands or worn as a backpack, and cannot be easily deployed. This absence of portability limits the use of ultrasound outside of a clinical setting. For example, emergency medical technicians, search and rescue professionals, and medics who must operate in conditions impacted by combat or natural disasters have urgent needs for medical imaging in order to better assess the nature and extent of injuries, but conventional ultrasound technology is not appropriate for the needs of those individuals because it is too difficult to carry in the field and deploy quickly.

The conventional means of making an ultrasound system more portable focus on reducing the size of at least one of the two constituent components. Systems still comprise a probe and a workstation, but the workstation or the probe or both are made smaller. However, the size of the workstation is still limited by the size demands of the display, user interface, and processing tasks. Making the workstation smaller reduces the surface area available to the user interface and/or the screen. The conventional response is to make the user interface smaller by simplifying or rearranging inputs. However, that can make the user interface difficult to use. Conventional thinking resists distributing those functions in different components. Additionally, while in a clinical setting multiple personnel are typically available to perform multiple roles, including operate the ultrasound system and obtain the images for a clinician who interprets them, a first responder or field medic must operate the system, obtain the images, and understand and interpret them, all while providing further assessment and treatment to a trauma patient, all in a potentially hostile environment. In order to be operable by a user under such circumstances, a system must minimize its impact on a user's mobility and the availability of a user's hands for other purposes. Finally, in a clinical setting ultrasound technicians are available who have received extensive training and/or have extensive experience with ultrasound systems. Field medics typically lack specialized expertise or extensive training in ultrasound. Therefore, an intuitive interface which accommodates the medic's various tasks and relative lack of specialized skills is very important. Finally, a field system must be self-powered, and make the most efficient use of available power possible in order to extend battery life.

Many of these requirements are competing. A wireless system avoids cables which interfere with a medic's mobility and other functions, but requires additional power and must accommodate additional components such as transmitters. An intuitive graphical interface which facilitates use by an individual who has not received extensive training in ultrasound use may require additional power, a larger footprint, a dedicated monitor, or may compromise the portability of the system in other ways. For these reasons, while ultrasound systems that purport to be portable are commercially available, they are not practical for use by military or other field medics, and ultrasound assessment of trauma patients in the field is still not possible with existing technology.

### SUMMARY OF THE INVENTION

Disclosed herein is a wearable ultrasound imaging system, comprising an ultrasound probe configured to emit and receive ultrasonic energy; a proximal wearable component electrically interconnected with said ultrasound probe, adapted to be wearable on the hand, wrist, or arm of a user, and including at least one user interface mechanism; a processor component adapted to be wearable on the body of said user, said processor component comprising at least one processor, said processor component electrically interconnected with said proximal wearable component, said processor component further comprising memory electrically interconnected to said at least one processor, said memory storing at least one set of instructions executable by said at least one processor; and one or more displays in communication with said processor; and wherein execution of said at least one set of instructions by said at least one processor causes said ultrasound probe to emit and receive ultrasonic energy in accordance with one of at least two sets of preset parameters, and said user interface mechanism is adapted to enable a user to select said one of at least two sets of preset parameters.

### BRIEF DESCRIPTION OF THE SEVERAL DRAWINGS

FIG. 1 is a view of one embodiment of the system described herein wherein the processor component and a display are interconnected wirelessly.

FIG. 2 is a view of one embodiment of the system described herein wherein a heads up display is used.

FIG. 3 is a view of one embodiment of the system described herein wherein the processing component and a display are interconnected with a cable.

FIG. 4 is a view of one embodiment of the system described herein wherein the processing component and the a display are wirelessly interconnected.

FIG. 5 is a view of one embodiment of the system described herein wherein said processing component is wearable on the arm of a user.

FIG. 6 is a rear perspective view of one embodiment of a finger mounted biplane ultrasound probe.

FIG. 7 is a lower perspective view of a finger mounted biplane ultrasound probe.

FIG. 8 is a perspective view of one embodiment of a proximal wearable component mounted on a user's wrist and interconnected with a finger-mounted probe using flex circuit.

FIG. 9 is a screenshot of one embodiment of a user interface, showing possible presets.

FIG. 10 is an example of a user interface which shows preset parameters corresponding to each scan in an e-FAST exam.

### DETAILED DESCRIPTION

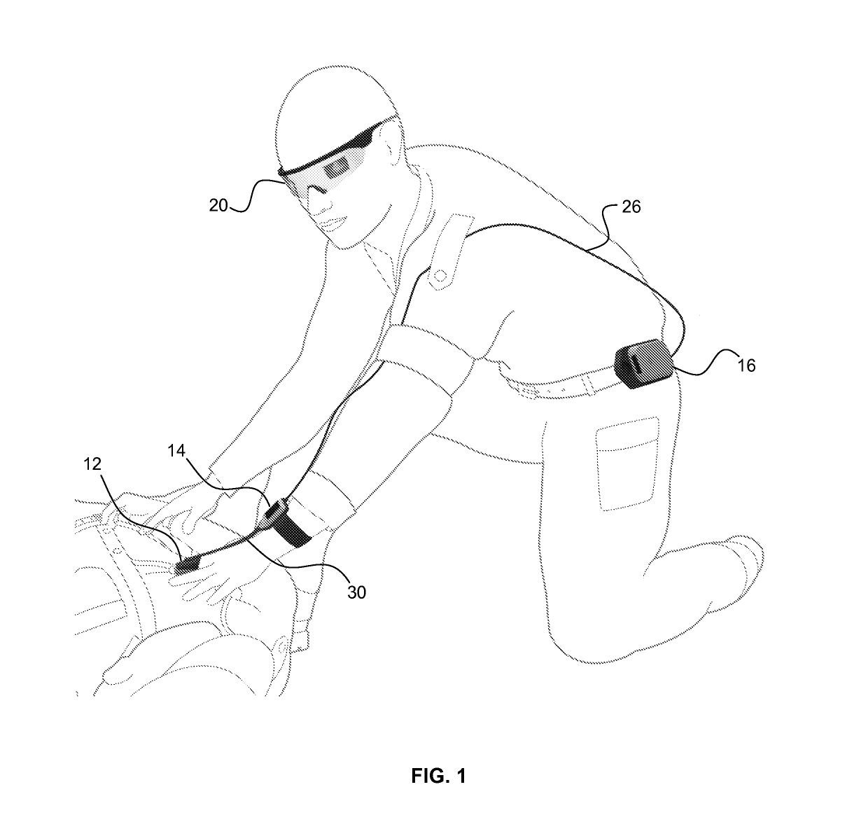

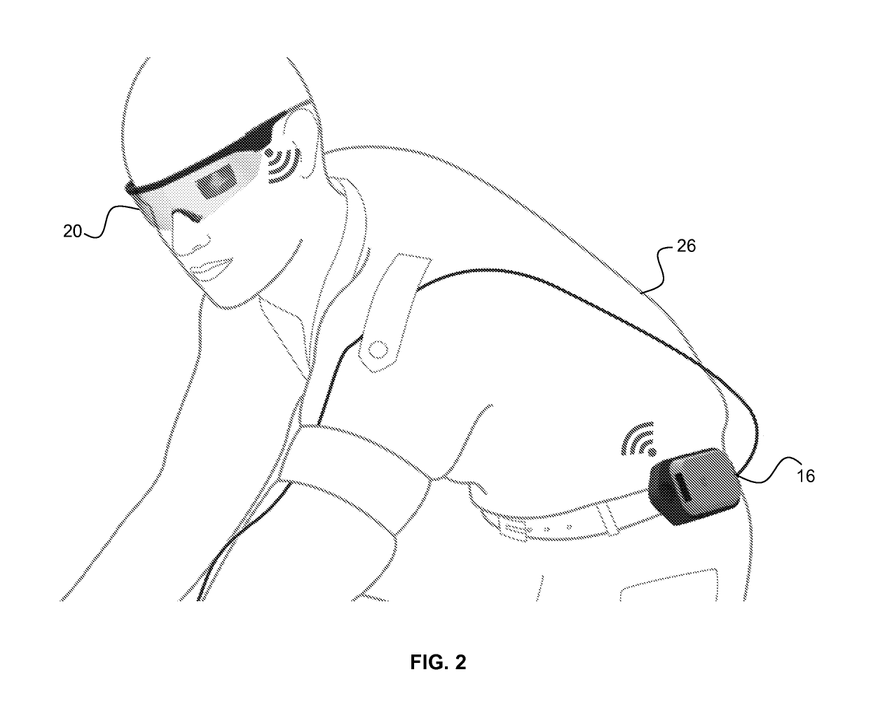

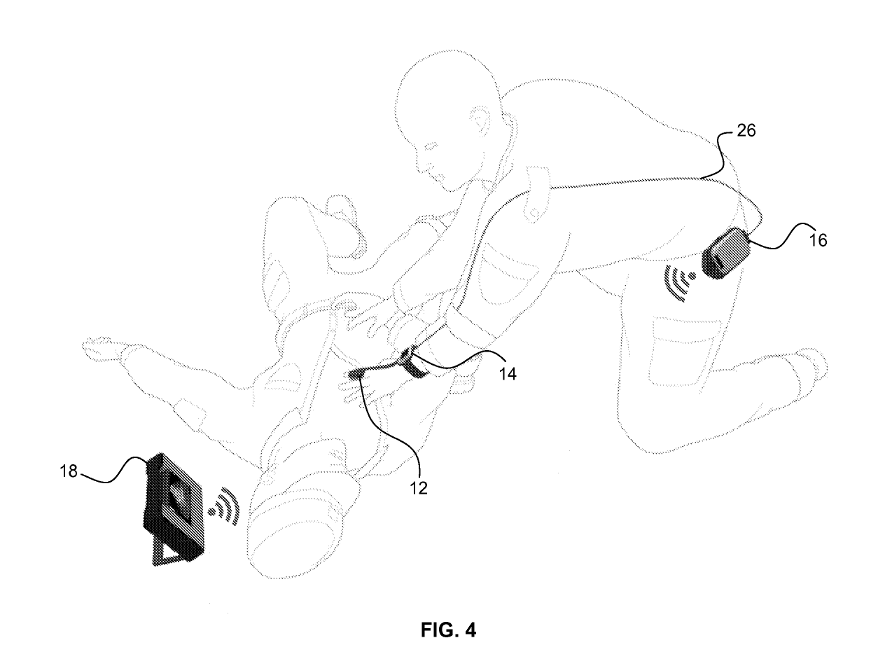

As shown in FIGS. 1-5, some embodiments of a wearable ultrasound system in accordance with the disclosure provided herein comprise four or more units: a light, small probe 12; a proximal wearable component 14 which can be attached to a user's wrist and preferably contains a multiplexor and user interface elements; a wearable processor component 16 which includes ultrasound front end and back end processing, and one or more portable displays 18, 20. The system may optionally also include a remote control (not shown) with wireless or wired connection with the processor and/or the display and which includes user interface elements and user input controls. Preferably, two or more system components include user input controls, which may be redundant.

The probe 12 includes a transducer consisting of an array of ultrasound elements, such as piezoelectric elements or a CMUT sensor, which convert electrical impulses into ultrasonic or acoustic energy and returning ultrasonic energy into electrical impulses which can be processed into images. A wearable system should ideally employ a light, small probe 12 which is easily deployed and the use of which is compatible with a medic's evaluation and examination of a patient. A finger mounted probe such as those disclosed in application Ser. No. 60/861,319 filed Nov. 27,2006 and application Ser. No. 10/863,644 filed Jun. 8, 2004, both of which are incorporated by reference as if fully set forth herein, or a sensor that is integral with a glove such as is described in U.S. patent application Ser. No. 13/645,317 filed Oct. 4, 2012 entitled GLOVE WITH INTEGRATED SENSOR would be particularly advantageous. However, a handheld probe may be used with the system disclosed and described herein.

Probes of various shapes and architectures permit varying field of views, and would be appropriate for use with this system. For example a curved linear array with relatively small radius of curvature permits imaging in the near field of the probe over a wide field of view. A phased array transducer permits imaging over a wide field of view at some distance from the array, while allowing imaging through a narrow access. A linear array permits imaging over a narrower field of view but provides good imaging of structures near the surface of the array. This is frequently the type of imagery that is highly desirable in surgical situations.

The use of a linear array in a finger mounted probe can be particularly advantageous. The probe can be configured so that the linear array images a scan plane that is parallel to the length dimension of the finger, or in another configuration, transverse to the finger. For the parallel configuration a portion of the scan looks forward from the finger, so that if the user directs his finger to point at the body surface, the probe will image a scan plane into the body. The user can then rotate the image plane by twisting his wrist, something that is quite easy for most users to do.

In the case of a curved linear array, the curved surface permits a user to rock the probe on the body or organ surface in order to view tissue over a variety of contact angles. This is particularly easy to do using a finger mounted probe, as the index finger has a good freedom of movement in several axes. The transverse mounted probe has the advantage that it permits a user to begin his examination with his hand transverse to the length of the patient's torso, which is a more natural position than parallel to the length of the patient's torso. A straight linear array or a phased array, however, has the advantage that the probe head profile can potentially be minimized, which is very important in accessing body portions.

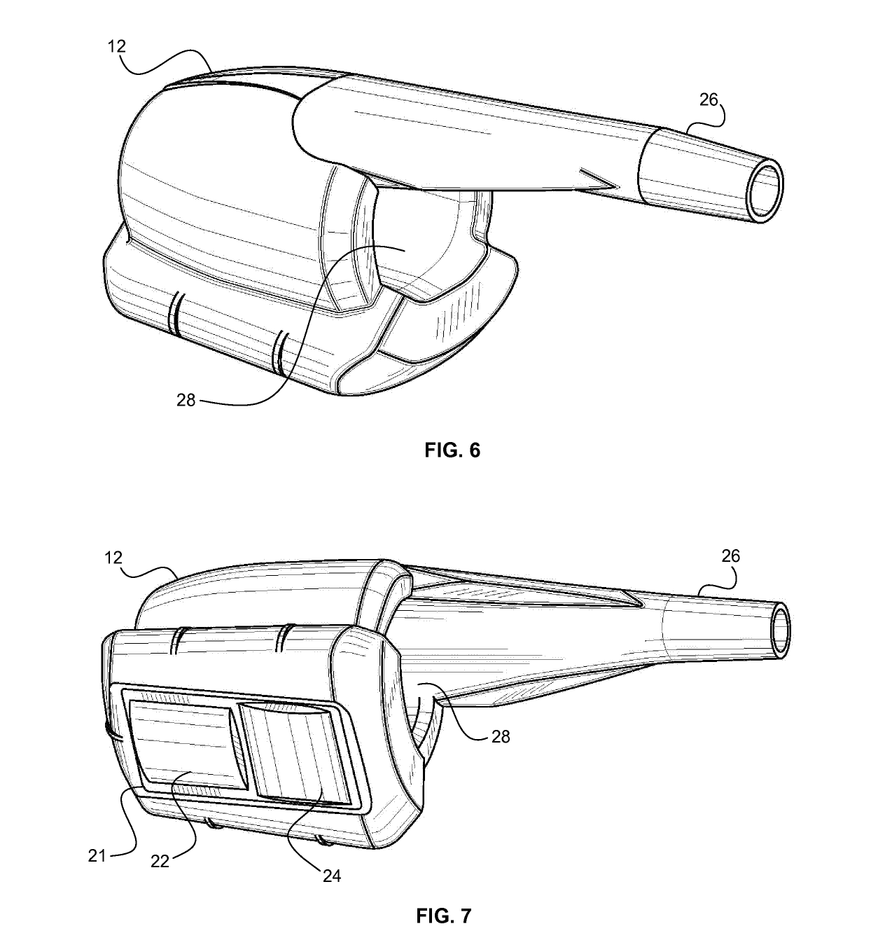

Especially advantageous in field use is a finger-mounted probe with a bi-plane array, such as the one shown in FIGS. 6-7. A bi-plane array 21 employs elements 22,24 arranged in both orientations to create a bi-plane probe capable of creating scan planes both parallel and transverse to the finger orientation. A user can select either parallel 22 or transverse 24 elements, and can toggle back and forth or use all elements sequentially to show both views simultaneously depending on preferences and scanning needs. A biplane probe is particularly advantageous in use of a finger-mounted probe to perform a FAST exam, as discussed below. A finger mounted probe is particularly advantageous for use by field medics who lack specialized ultrasound expertise because the ergonomic form of the probe leverages innate hand-eye coordination to simplify use and training. Additionally, a finger mounted probe helps keep a user's hand and arm available for other uses.

Any such finger mounted probe may preferably have an array 21 which is electrically interconnected with a cable 26 on an aspect of the probe opposing the array. A structure (not shown) which can be made of flex circuit or any other electrically conductive or connective material may be employed to electrically interconnect the array and the cable in a way that permits the cable to exit or extend from the probe at an opposing aspect of the housing from the elements. Such a structure may encircle a receptacle 28 for a user's finger which is defined by the housing so that the sensor may be placed beneath the pulp of the user's finger. Such a structure is described in U.S. patent application Ser. No. 13/525,078 filed Jun. 15, 2012 entitled PROBE WITH DORSAL CONNECTIVITY.

Ultrasound probes are in contact with patients during use, and therefore need to be sanitized or even sterilized or else enclosed in sterile or sanitary sheathing. This sheathing may interfere with access to any user interface elements which are located on the probe. For this reason, conventional ultrasound systems do not typically offer user interface components associated with the probe or located elsewhere than the processor or display. However, the processor and/or display may be difficult to reach under some circumstances.

Hand held probes which are larger in size tend to include additional functionality and components beyond the sensor array. For example, conventional probes can include transmit and receive beamformers, a beamformer control unit, and digitizing, and amplification functionality. Smaller, lighter probes such as finger mounted probes can generally accommodate only the sensor array and connectivity, however, which means that components which perform these additional functions must be housed elsewhere in the system.

A robust connection between the transducer and the beamformer and other functional components is necessary if those components are not located in the same unit as the transducer is. However, that connection must not interfere with the mobility and capability of a user in the field. A wireless connection would not interfere with the user. However, a wireless transmitter and sufficient power source cannot be accommodated by a very small, light probe. Consequently, a small, light transducer may preferably be connected to other system components by a cable, which can be cumbersome in any context and which can be especially disruptive to the activities of a medic functioning in the field.

In accordance with some embodiments of the system disclosed herein, a small, light probe 12 is interconnected using a connector 26 such as a cable or flex circuit as shown in FIG. 1 with a proximal wearable component 14 which is located in fairly close proximity to the probe 12 or the hand using the probe. This component need not be attached to the wrist but could be worn on the arm or hand. The proximal wearable component 14 may preferably contain a multiplexor which reduces the number of channels which must be accommodated by connectivity to other components of the system. For example, the wired connection 26 between the probe and this component may contain 128 channels. The multiplexor contained in the proximal wearable component 14 may reduce the number of channels necessitated by wired connections downstream from the proximal wearable component to 64 channels, in addition to USB and power. Optionally, the proximal wearable component 14 may include some front end functionality.

The proximal wearable component 14 as described above is electrically interconnected with a processor component 16, which contains one or more processors, in addition to beam former functionality and other ultrasound front end, mid end, and back end functionality. The processor component can be worn on a user's arm, hip, or shoulder. It can be connected to the wrist component wirelessly. However, because of the multiplexor which is preferably present in the proximal wearable component 14, synthetic beamforming, and other means of limiting the number of channels that must be included between the proximal wearable component and the processor, a relatively small cable 30 or flex circuit or other connector can be effectively used to interconnect these components in a way which does not unduly interfere with activities of the user. Such a cable is shown in FIGS. 1-5. Alternatively, the connection may be wireless.

A beamformer emits the electrical pulses which are transformed into ultrasonic energy by the probe and used to image the patient or substrate. The beamformer originates the signal, and times it in order to focus the acoustic beam that emits from the transducer. The beamformer determines the amplitude and frequency of the signal. The beamformer also receives the signal and demodulates, filters, detects, and compresses the signal and converts ultrasound data into pixels, or processed image information which can then be converted to a video stream and fed to the display.

Synthetic beamforming may be used in some embodiments of the system disclosed herein. Synthetic beamforming generates ultrasound images by archiving several transmit-receive events which are then coherently summed to form a synthetic beam. The inventors of the system described herein have used synthetic beam forming to generate diagnostic quality images at up to 20 cm depth at 10 frames per second with a 32 channel transmit and 16 channel receive stepped synthetic aperture.

Because a beamformer cannot be located in a very small, light probe, it should be placed either in the proximal wearable component 14 or in a processor component 16.

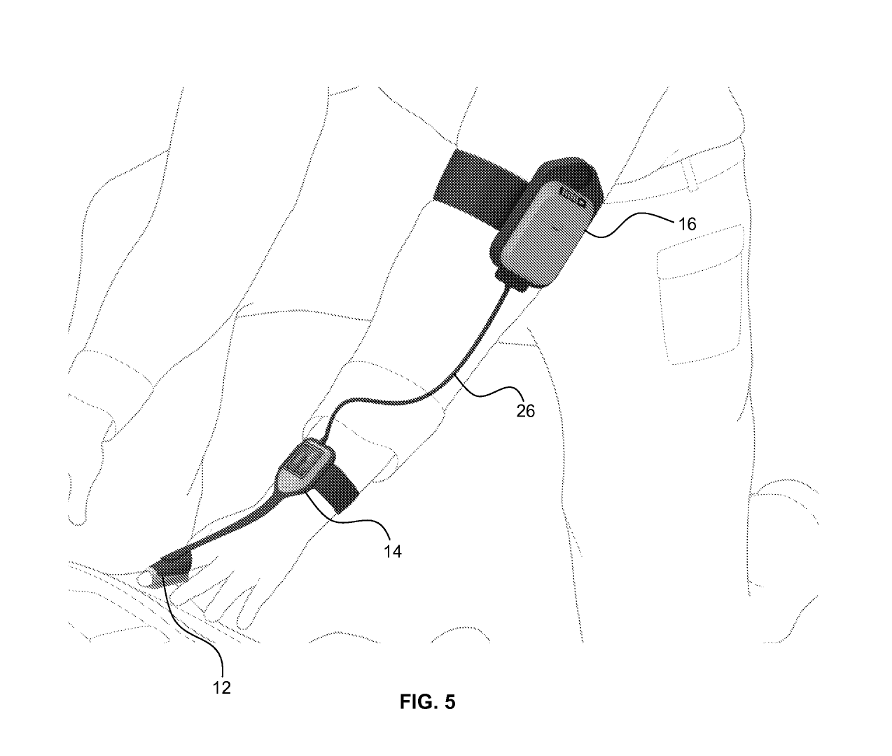

In accordance with some embodiments of the system disclosed herein, the processor component 16 should be wearable on a user's arm, as shown in FIG. 5, hip, waist, shoulder, or elsewhere on a user's body. The processor component 16 includes one or more processors. The processor component may include ultrasound front end functionality. It may include the transmit/receive switch, amplification, digitization, beamformer, connection capability such as wi-fi, Ultra Wide Band, or USB. Finally, the processor component stores and executes instructions supplied by the ultrasound operating system which directs the performance of the system.

The ultrasound system disclosed herein is controlled by software which includes instructions to implement various operations recorded in non-transitory computer readable media. The instructions make up an operating system which directs the system to perform operations associated with system set up, system control, scanning, data acquisition, beamforming, signal processing, and image creation. The operating system may include data files and data structures in addition to program instructions. The processors include memory consisting of hardware specially configured to store and perform program instructions such as the operating system and to record and store data and images generated by the system.

The processing component further includes an actual or virtual control component, which receives signals generated by user interface elements on the processing component and other components of the system and alters the action of the beamformer, display, processors, and/ or other components in order to conform the performance of the system with the user input. For example, it may alter the screen brightness of the monitor to change. It may also alter system performance in accordance with preset scanning parameters as described below.

The processor component 16 may include user interface elements. Any such user interface elements should preferably be tactile in nature. For example, toggle switches or buttons with differentiating features that can be detected by touch would enable a user to operate the user interface without looking at the processor component. It should include a power source which is used to power the processor and may preferably also provide power to the probe and the proximal wearable component located proximal to the probe. Optionally, it may also power one or more display components.

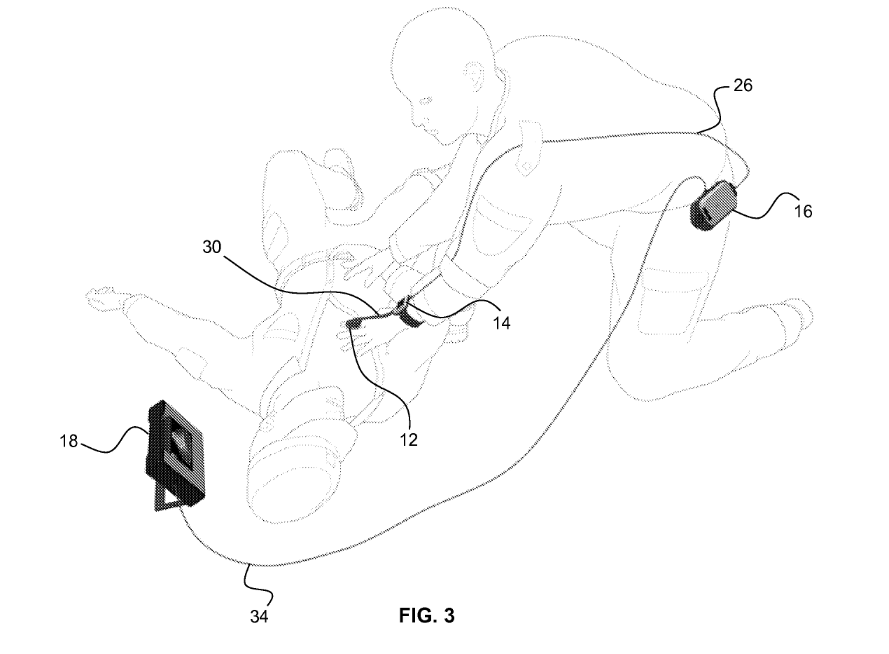

The system comprises one or more portable displays 18, 20 which can include units which include a screen and which may be positioned on the ground within view and reach of the user. The display may be connected to the processor component 16 with a wireless (as shown in FIGS. 1, 2, and 4) or wired connection (as shown in FIG. 3) which accommodates the transmission of video display data and a two way USB link for control of the system. The display may contain a user interface, which may take the form of a touch screen, buttons, toggles, or any other interface elements known in the art. These interface elements are preferably at least partially redundant of those elements which are present on the proximal wearable component and/or processor component in order to accommodate different user preferences. The display 18 may include one or more processors, memory, and software which directs the processor to change the function of the display and/or the system in response to user input either via user interface elements present on the display or user input conveyed through user interface elements elsewhere in the system.

The system may also include a heads up display 20, and displays which are located remote from the user and visible to individuals who are not present at the site of examination, such as a doctor in a command center of field hospital. Remote control of the system may also be facilitated.

Ultrasound scanning is subject to variable parameters, and manipulation of those parameters enables users to optimally image structures located at various depths within a substrate such as a patient's body. Ultrasound system user interfaces typically have some or all of the following user inputs: a power switch, an ability to adjust the array, an ability to adjust the gain, or brightness or vividness of the signal, an ability to optimize images, and a zoom capability. A bi-plane probe should be interconnected with a user interface which enables a user to change the scan plane. Battery change indicators, screen brightness and contrast, and cine arrows to move between images are also important features. Finally, ultrasound system user interfaces typically allow users to freeze images and to save or record images or video.

Additionally, most ultrasound systems include presets, which are used to set standardized parameters for standardized scans. The extended, Focused Assessment using Sonography in Trauma (eFAST) exam is a universally accepted triage and rapid assessment tool based on a rapid ultrasound survey of key organs, internal bleeding, and heart and lung function. The FAST protocol involves serial scans: The subxiphoid four chamber view and the parasternal long axis view of cardiac anatomy; abdominal and lower thoracic views including the upper peritoneum and Morison's pouch between the liver and right kidney and the lower peritoneum posterior to the bladder in the male and the pouch of Douglas (posterior to the uterus) in the female; right coronal and intercostal oblique views in the mid-axillary line giving coronal views of the interface between the liver and kidney; left coronal and intercostal oblique views from the posterior-axillary line producing coronal views of the spleen and diaphragm; longitudinal and transverse lower pelvic views of the bladder (male/female) and uterus (female); and anterior thoracic views of the pleural interface (to access pneumothorax) through the 3-4th intercostal space and midclavicular line.

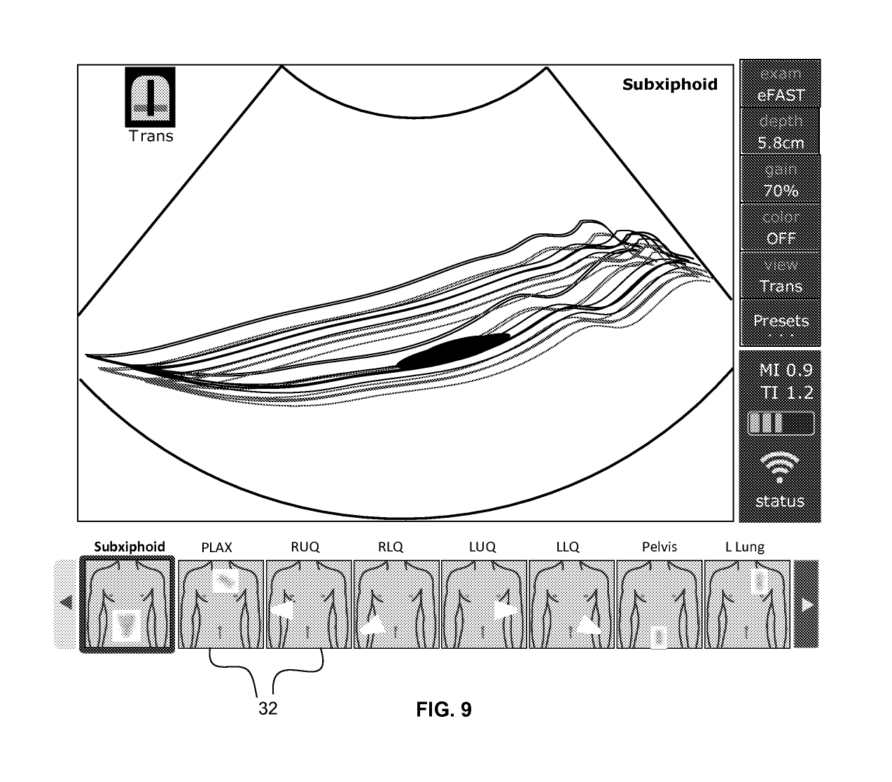

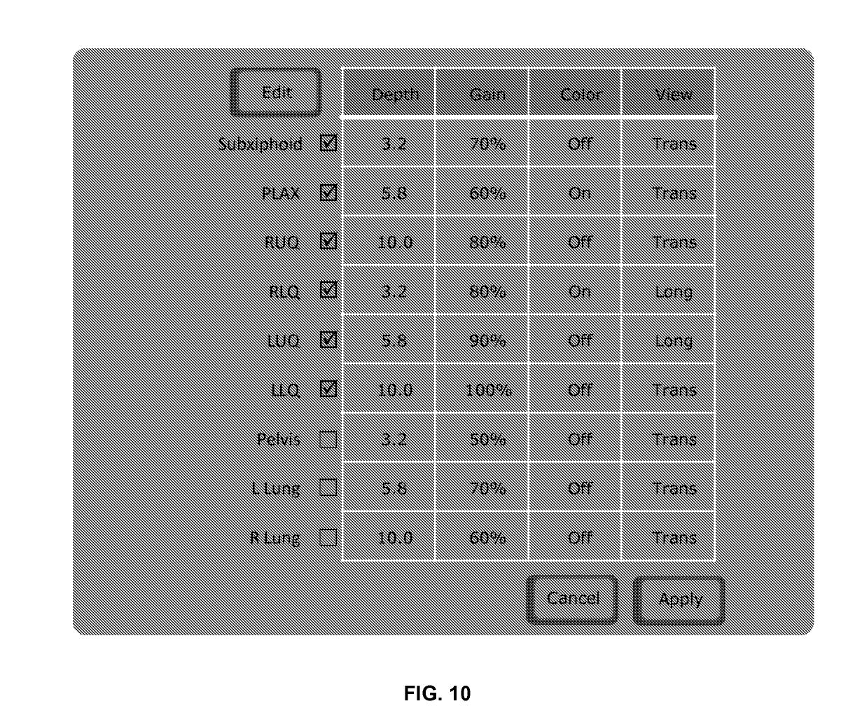

An e-FAST examination is facilitated by preset parameters most appropriate for each successive scan, e.g., gain, depth, scan plane, and other system parameters optimized for each area of the body scanned during an e-FAST exam, pre-programmed into the system and categorized by scan. An example of a user interface which shows preset parameters corresponding to each scan inane-FAST exam is shown in FIG. 10. A user can initiate an eFAST exam, causing the system to automatically set system parameters optimized for the first scan in accordance with the first pre-set. When a user has completed that scan, the user so indicates to the system, which saves the scan and then changes system parameters so that they are optimized for the next scan in accordance with the next pre-set, and so on. An illustrative user interface is shown in FIG. 9. Icons 32 which represent each scan in an e-FAST exam permit a user to indicate which scan he or she would like to perform. In response to that indication, the system is automatically configured to scan in accordance with the preset parameters associated with that scan. Preset scan parameters mean that users need not adjust individual parameters when transitioning between scans. Instead, users merely transition between preset parameters as they transition between scans.

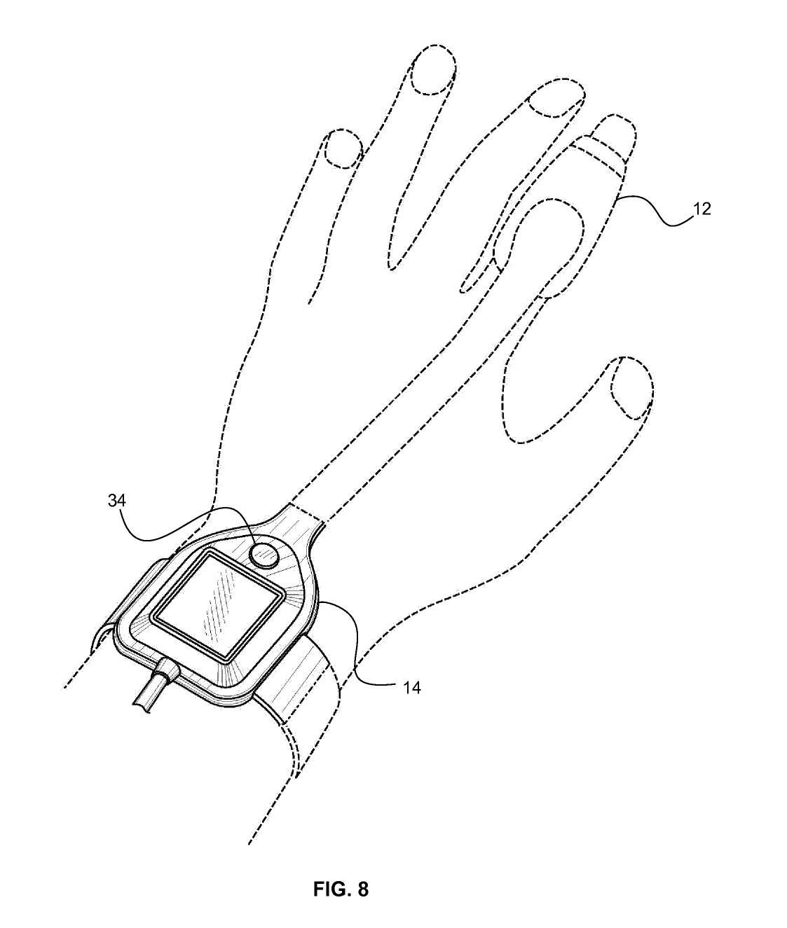

One example of one embodiment of a proximal wearable component is shown in FIG. 8. The proximal wearable component 14 should contain a few-preferably fewer than four-buttons 34 which permit a user to switch between preset parameters, freeze, and save images, and in accordance with some embodiments of the system disclosed herein change scan plane and/or adjust gain and depth. The proximal wearable component should also preferably include a screen 34 or display which reflects system parameters and may be a touch screen which can accept user input.

Alternatively and preferably, a user can provide an input through a movement of his or her wrist which causes a switch between preset parameters and optionally also saves the scan data which was gathered immediately preceding the input. Optionally, the system may freeze the image created immediately or a predetermined number of seconds prior to the input, and then save that data and move to the next set of preset scanning parameters in response to an additional user input. It is understood that the system may save scan data continuously, and either selectively erase data or write over data or selectively preserve data from being erased or written over in response to user input.

Motion sensors can include accelerometers, which measure acceleration in one to three linear axes, gyroscopes, which measure angular velocity, and IMUs or inertial measurement units, which typically contain both gyroscopes and accelerometers. The inclusion of one or more motion sensors in the proximal wearable component would enable a user to shake, jerk, or simply move his or her wrist in order to provide user input into the system.

By way of illustration, a user would instruct the system to move through a FAST exam by flicking his or her wrist at the conclusion of each scan. The system would establish the first set of preset parameters, the user would perform the scan, and then flick his or her wrist at the conclusion of that scan once a satisfactory image had been obtained. The motion sensor in the proximal wearable component would detect the movement of the unit and provide a signal to the system to save the preceding images and change the scan parameters in accordance with the next set of preset parameters. At the conclusion of each scan, the user's wrist movement would cause the system to save data and move to the next scan until the FAST exam was complete. If a user did not experience a need to adjust the preset parameters, the user could complete a FAST ore-FAST exam without providing any user input via buttons between scans, would not need to take his or her eyes off the patient or the display, and would not need to touch the display or any other user interface, which would minimize the risk of contaminating ultrasound system components with blood, other bodily fluids, or other contaminants.

In one embodiment, the proximal wearable component 14 includes a motion based sensor which has responsiveness which is limited to certain directions. For example, an accelerometer which measures acceleration in one or two linear axes would be suitable for this purpose. Motions made by the user in the course of examination which are not intended by the user to be an input would be less likely to cause the sensor to register a movement sufficient to constitute a user input. Alternatively, the sensitivity of the sensor may be set so that a movement that exceeds a certain magnitude is necessary in order to cause the sensor to register a movement sufficient to constitute a user input.

Other presets may be used within the spirit and scope of the system disclosed herein. For example, presets may be defined by the area of the body to be imaged, for example, breast, spleen, bladder, etc.

One or more microcontrollers associated with the motion sensor should preferably be located within the proximal wearable component 14. Data from the microcontroller may be sent to the a processor which may be located in the processor component 16 in the form of a signal, in order to effectuate a change between preset scanning parameters. Alternatively, a parameter change command may originate in the proximal wearable component.

One or more embodiments of the system disclosed herein may include user interface elements that are redundant with respect to the user interface elements on the proximal wearable component. For example, user interface elements may be present on the proximal wearable component 14 and on a display 18, 20, and a user may be able to effectuate the same change in system function with user elements present on either unit, which enables the system to accommodate user preferences. At times during an examination, the proximal wearable component 14 may not be accessible because the user's wrist is obstructed by the patient or blankets, armor, etc. In such cases a user may accomplish the same objectives by using interface elements on the display. It is also understood that the display or the wrist unit may be or include a touch screen or may include dedicated buttons, track balls, touch pads, or other types of user interface elements known in the art.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A wearable ultrasound imaging system, comprising:
a) an ultrasound probe configured to emit and receive ultrasonic energy;
b) a proximal wearable component electrically interconnected with said ultrasound probe, adapted to be wearable on the hand, wrist, or arm of a user, and including at least one user interface mechanism;
c) a processor component adapted to be wearable on the body of said user, said processor component comprising at least one processor, said processor component electrically interconnected with said proximal wearable component, said processor component further comprising memory electrically interconnected to said at least one processor, said memory storing at least one set of instructions executable by said at least one processor; and
d) one or more displays in communication with said processor; and
e) wherein execution of said at least one set of instructions by said at least one processor causes said ultrasound probe to emit and receive ultrasonic energy in accordance with one of at least two sets of preset parameters, and said user interface mechanism is adapted to enable a user to select said one of said at least two sets of preset parameters.

2. The ultrasound system of claim 1 wherein said display is wearable on said body of said user.

3. The ultrasound system of claim 1 wherein said display is not adapted to be wearable on the body of said user.

4. The ultrasound system of claim 1 wherein said ultrasound probe and said proximal wearable component are interconnected using flex circuit.

5. The ultrasound system of claim 1 wherein said ultrasound probe and said proximal wearable component are interconnected using cable.

6. The ultrasound system of claim 1 wherein said proximal wearable component further contains a multiplexor.

7. The ultrasound system of claim 1 wherein said proximal component and said processor are interconnected wirelessly.

8. The ultrasound system of claim 1 wherein said processor component and said display are interconnected wirelessly.

9. The ultrasound system of claim 1 wherein said display comprises a user interface mechanism, and said user interface mechanism on said display and said user interface mechanism on said proximal component are at least partially redundant.

10. The ultrasound system of claim 1 wherein said user interface mechanism on said proximal wearable component comprises a motion sensor.

11. The ultrasound system of claim 1 wherein said proximal wearable component is adapted to be worn on the wrist of a user.

12. The ultrasound system of claim 11 wherein said user interface mechanism on said proximal wearable component is adapted to permit a user to select one of said at least two preset parameters by moving said user's wrist.

13. The ultrasound system of claim 1 wherein said processor component comprises a user interface mechanism and said monitor contains a user interface mechanism and said user interface mechanisms are at least partially redundant.

14. The ultrasound system of claim 1 wherein said ultrasound probe is a finger mounted probe.

15. The ultrasound system of claim 1 wherein said ultrasound probe is adapted to be mounted on a finger during use.

16. The ultrasound system of claim 1 wherein said ultrasound probe is a biplane probe.

17. The ultrasound system of claim 15 wherein said ultrasound probe comprises an element adapted to create a scan plane that is transverse to said finger and an element adapted to create a scan plane that is parallel to said finger.

18. The ultrasound system of claim 1 wherein said preset parameters comprise depth and gain.

19. The ultrasound system of claim 18 wherein said preset parameters further comprise scan plane.
